(19) United States
(12) Patent Application Publication
(10) Pub. No.: US 2012/0310274 A1
(43) Pub. Date: Dec. 6, 2012

(54) METHOD OF PERFORMING MULTIPLE ORAL AND NASAL SURGICAL PROCEDURES

(76) Inventor: ABDULMOHSEN E.A.H. AL-TERKI, MISHREF (KW)

(21) Appl. No.: 13/152,048

(22) Filed: Jun. 2, 2011

(51) Int. Cl. A61B 17/03

(52) U.S. Cl. 606/213

(57) ABSTRACT

The method of performing multiple oral and nasal surgical procedures may be applied to various different but related medical procedures, but is particularly well adapted for the performance of multiple operations for correcting various ear, nose, and throat conditions that cause obstructive sleep apnea (OSA) in the patient. The method allows the surgeon to perform multiple operating procedures in the nose, mouth, and/or throat in a single operating session, due to the use of hemostatic sheet and gel treatments in lieu of suturing and post-operative nasal hemostatic packs that preclude nasal breathing. A tracheal breathing tube is placed in the patient during the operation, leaving the surgeon free to perform multiple operations in the nose, mouth, and/or throat with hemostatic packs being placed immediately following each specific operating procedure. The packs are removed at the end of the operations before removal of the tracheal tube.

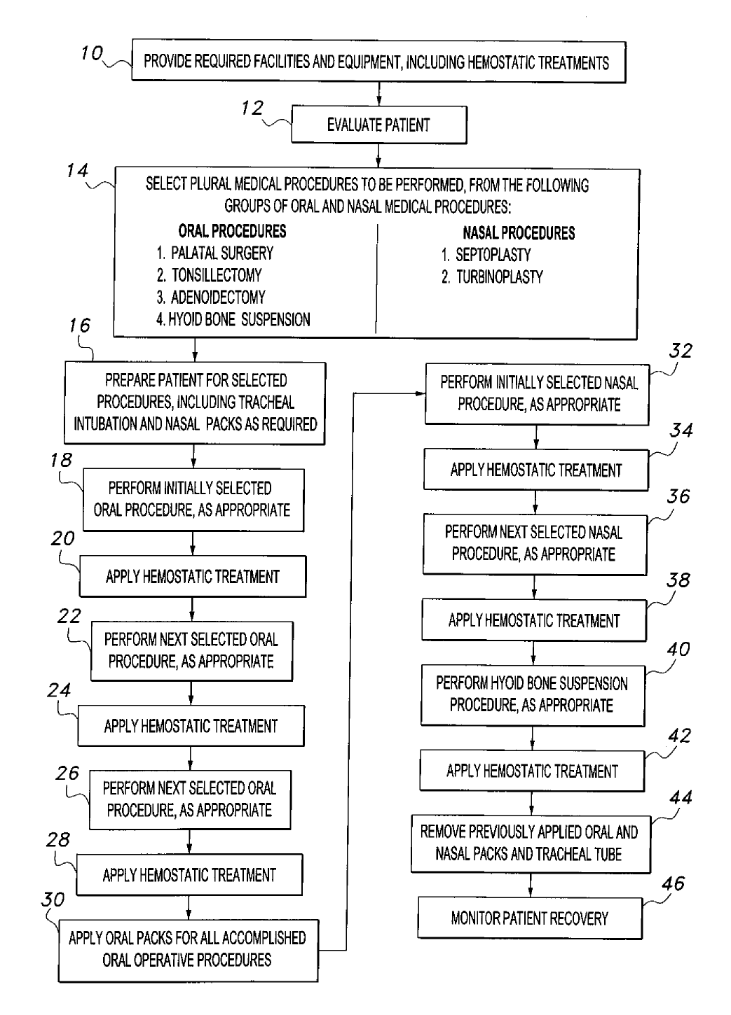

### BACKGROUND OF THE INVENTION

1. Field of the Invention

The present invention relates generally to the medical and surgical field, and more particularly, to a method of performing multiple oral and nasal surgical procedures that allows the surgeon to perform the multiple oral and/or nasal surgeries during a single operative session.

2. Description of the Related Art

Ear, nose, and throat (ENT) surgery is a very important specialty in the medical field. The surgeons and medical professionals specializing in this field are highly trained and skilled in order to handle the multiple disciplines required. While an ENT surgeon may be called upon to perform only a single operation on any one of the above organs, his or her specialty is often needed to perform multiple operations on two or more of the ear, nose, and throat organs. An excellent example of a situation in which multiple ENT operations may be required is for a patient suffering from obstructive sleep apnea (OSA), where it may be necessary to perform one or more operations to improve the air path through the mouth, throat, and nose of the patient.

In the past it was necessary for the ENT surgeon to perform a single operative procedure upon one of the subject organs (e.g., the nose) and allow time for patient healing before performing a second operation upon another organ (e.g., the mouth and/or throat). This was due to the need to place hemostatic packs of absorbent cotton or other suitable material upon the sutured or cauterized incision after the operation to stop any flow of blood that might occur. The packs would remain in place for perhaps one to two days. Obviously, a surgical procedure performed in the nose with hemostatic packs remaining in place for an extended period would require the patient to breathe through his or her mouth until the packs were removed. If an additional surgical procedure were performed on or in the mouth or throat (e.g., tonsillectomy or adenoidectomy), the irritation to the throat due to breathing through the mouth would be exceedingly uncomfortable for the patient, and might even be impossible, depending upon the throat restriction due to post-operative edema and the installed hemostatic packs.

Accordingly, it has been standard procedure to perform a single operation in one operative session, and then wait for the patient to recover before performing a subsequent operation. Clearly, this results in additional trauma to the patient and considerable additional cost, as many such procedures are sufficiently involved as to require hospitalization, at least on an outpatient basis, if not overnight. Such multiple operations also result in lower efficiencies for the hospital as well, as the hospital must arrange for and release the same patient for each surgical procedure performed. The surgeon must arrange for an operating room and staff for each surgery, as well.

Modern medicine has resulted in a number of improvements in operative and post operative care for patients in terms of equipment, procedures, and treatments. An example of such is found in relatively recently developed hemostatic materials or treatments that may be applied to an incision to stop bleeding, rather than the older cotton or gauze packs commonly used. Examples of such treatments are the Surgicel® hemostatic sheet material and Surgiflo® hemostatic fluid (Surgicel and Surgiflo are both registered trademarks of Johnson & Johnson Corporation, of New Brunswick, N.J.), both produced by the Ethicon Company (a subsidiary of Johnson & Johnson). Other similar products are produced by other companies, as well. These treatments serve to replace the older hemostatic packs and the like, and may include analgesic and antibiotic properties as well. Many of these products are absorbed into the body after some period of time, thus precluding any requirement for removal, as would be required with stitches.

However, the problem of multiple procedures on separate occasions remains. Thus, a method of performing multiple oral and nasal surgical procedures solving the aforementioned problems is desired.

### SUMMARY OF THE INVENTION

The method of performing multiple oral and nasal surgical procedures in a single operating session enables an ear, nose, and throat (ENT) surgeon to perform multiple procedures in different organs (e.g., the nose, mouth, and throat) in a single operative session. This provides great savings in time and expense for the patient, as well as saving time for the surgeon and hospital or medical facility.

The multiple surgical procedure method is enabled by the use of hemostatic sheet material and gel, rather than conventional sutures or cauterizing of the incisions. This greatly reduces the need for conventional hemostatic packs of cotton or gauze material, which conventionally remain at the site of the incision for up to two days. Clearly, the placement of hemostatic packs in the nasal cavities for a prolonged period requires the patient to breathe orally. This generally precludes the performance of oral surgery in conjunction with nasal surgery in a single operating session. The present method of performing multiple operations in a single operating session overcomes this problem through the use of hemostatic sheet and/or gel treatments, which do not obstruct airflow through the nose. This allows the surgeon to perform multiple medical procedures in the nose, mouth, and/or throat in a single operating session, thus avoiding the problems of multiple patient trauma over perhaps months of operations, the need to schedule multiple hospital visits, and the need to schedule multiple operating room sessions and staff for those sessions.

The method of performing multiple surgical procedures may be applied to a number of different but related medical procedures, but is particularly well suited for the treatment of conditions causing obstructive sleep apnea (OSA). This problem can be due to a number of different conditions occurring in the nose, mouth, and throat, and it is not uncommon for a patient to require multiple operations to correct various ones of these conditions. These conditions may comprise a deviated nasal septum, tonsil and adenoid enlargement and/or infection, hypertrophy of one or both of the inferior turbinate nasal passages, a weak or flaccid soft palate structure, and/or a poorly located hyoid bone. While it would be extremely rare for any one individual to have all of these conditions, it is quite common for an individual suffering from OSA to have two or more such conditions.

According to the present method, the surgeon initially evaluates the patient to determine which of several procedures are needed. A single surgery session is scheduled and the surgeon performs the individual operations as necessary, using hemostatic sheet and/or gel treatments in order to preclude long-term blockage of the nasal and/or oral passages of the patient. A tracheal tube is installed in the patient for the duration of the operations, to allow the patient to breathe during the operations when the nasal cavity is packed with hemostatic material. The hemostatic packs are removed at the end of the surgery or following the completion of a given surgical procedure. This allows the patient to breathe freely even though multiple surgical procedures may have been performed in both the nasal and oral passages.

These and other features of the present invention will become readily apparent upon further review of the following specification and drawings.

### BRIEF DESCRIPTION OF THE DRAWINGS

The sole drawing FIGURE is a flowchart briefly describing the basic steps in the method of performing multiple oral and nasal surgical procedures according to the present invention.

Similar reference characters denote corresponding features consistently throughout the attached drawings.

### DETAILED DESCRIPTION OF THE PREFERRED EMBODIMENTS

The method of performing multiple oral and nasal surgical procedures enables a surgeon to accomplish a series of operations in a single operating session, thereby greatly reducing trauma and cost for the patient and saving considerable time for the surgeon and patient. While the present disclosure is directed primarily to operations involving ear, nose, and throat (ENT) surgery, it will be seen that the method may be expanded to other groups of closely related surgical operations and procedures. The method employs the application of hemostatic surgical gel and/or hemostatic surgical sheet material to the surgical incisions immediately following the completion of each specific surgical procedure in lieu of suturing or cauterizing the incision, thereby greatly reducing trauma to the patient and the need to retain hemostatic packs in the nasal or oral cavities of the patient for some time after the operation(s).

The sole FIGURE provides a flowchart briefly describing the steps of the method. As in all medical procedures, the initial step is the provision of the required facilities and equipment, as generally indicated by the first step 10. The hemostatic treatments noted in the first step 10 may include an applicator preloaded with a hemostatic surgical gel and hemostatic sheet material, in addition to hemostatic packs of cotton, gauze, and/or other suitable materials. All of the above hemostatic treatments are conventional. An example of a hemostatic surgical gel and applicator is Surgiflo® hemostatic matrix, produced by the Ethicon Company. An applicator is also provided for preloading with the Surgiflo material, the applicator having a choice of cannulas or “straws” for applying the material accurately to the site of the incision. An example of a hemostatic sheet material is Surgicel®, an absorbable cellulose-based material produced by the Ethicon Company. The Surgicel® material may be cut to size for application to the incision or other surgical wound. Both the Surgiflo® and Surgicel® materials may also be treated with analgesic and/or antibiotic agents, if desired. These materials and their treatments are conventional, as noted further above.

The patient is evaluated, as generally noted in the second step 12 of the flowchart. The evaluation may comprise physical examination and/or radiological examination (e.g., x-ray), as appropriate. From this evaluation, the surgeon determines the specific surgical procedures or operations to be performed during the single operation session. The present method is applicable when the surgeon will perform at least two of the surgical procedures. These surgical procedures may comprise at least one oral and/or tracheal procedure and at least one nasal procedure, or may comprise plural oral procedures and no nasal procedures, or plural nasal procedures and no oral procedures. The specific procedures to be performed are at the discretion of the surgeon, and will depend upon the condition of the patient as determined by the evaluation.

The third step 14 of the chart includes a general list of the oral and nasal procedures that may be performed by an ear, nose, and throat (ENT) surgeon, the listed procedures being exemplary of those generally performed for treating obstructive sleep apnea (OSA). It will be understood that the general method of performing two or more closely related surgical procedures and treating the incisions with non-obstructive hemostatic materials may be expanded to other areas, in addition to those listed. In the case of an ENT surgeon treating a patient suffering from OSA, the surgeon would select a plurality of procedures from the list of oral procedures comprising palatal surgery, tonsillectomy, adenoidectomy, and hyoid bone suspension surgery (the latter procedure might more properly be considered tracheal surgery). The palatal surgery preferably comprises the placement of Pillar® implants (Pillar is a registered trademark of Medtronic Xomed, Inc. of Jacksonville, Fla.) in the soft palate to stiffen the soft palate, but may comprise other procedures, e.g., uvulectomy or uvulopalatopharyngoplasty (UPPP), as desired by the operating surgeon. The list of appropriate nasal procedures is somewhat shorter, comprising only septoplasty (for a deviated septum) and turbinoplasty (to open the turbinate nasal passages). Again, the ENT surgeon may select a plurality of procedures solely from the oral and tracheal procedures or solely from the nasal procedures, or from both oral and nasal procedures, depending upon the needs of the patient.

At the time of the scheduled operations, the patient may be prepared for surgery, generally as indicated by the fourth step 16 of the flowchart. While some of the oral and nasal operations included in the present method have been conventionally performed under local anesthesia while the patient remains conscious, the patient is placed under general anesthesia and is unconscious for the multiple procedures of the present method, since at least some of the operations conventionally require general anesthesia. As both the oral and nasal breathing passages may be closed simultaneously during the surgical procedures, a tracheal tube is placed in the patient to allow the patient to breathe during the surgical process. Other steps in the preparation process may include the placement of nasal packs having antibiotic and/or analgesic properties, as required.

At this point the patient has been prepared for surgery, including placement under general anesthesia and tracheal intubation. The surgeon begins the process by performing the first oral operation as appropriate, generally as indicated by the fifth step 18 of the flowchart. The first oral operation is selected from the group of oral operations noted in the third step 14 of the flowchart. The first oral operation may be the palatal surgery, which may comprise one of a few different surgical techniques or procedures. For example, the surgeon may elect to perform uvulectomic or uvulopalatopharyngoplastic (UPPP) surgery to remove the uvula and/or generally improve the palatal and pharyngeal processes of the patient. However, a more recently developed process, comprising the placement of Pillar® implants in the soft palate to stiffen the soft palate, has been found to produce excellent results. This Pillar® implantation procedure has conventionally been accomplished using a local anesthetic, but the patient is placed under general anesthesia for the present multiple operation method due to the need for general anesthesia for certain other operations that may be performed under the method.

When the initial operation has been completed, a hemostatic treatment is applied to the area, generally as indicated by the sixth step 20 of the flowchart. The hemostatic treatment is in the form of a hemostatic gel, e.g., Surgiflo®, as noted further above, for hemostatic treatment of the Pillar® implantations or other palatal surgery. The hemostatic gel is applied by means of a preloaded applicator having a tube or cannula for precise placement of the gel at the desired location, as noted further above. In some instances a hemostatic sheet material, e.g., Surgicel®, may be applied in lieu of the hemostatic gel. Both the hemostatic gel and the hemostatic sheet material may include other additives to provide analgesic and/or antibiotic properties, if desired.

At this point the surgeon moves to the next planned oral operation, generally as indicated by the seventh step 22 of the flowchart. The second oral operation is selected from the remaining oral operations of the list of the second step 14 of the flowchart. Assuming the patient's tonsils are enlarged and still in place, a tonsillectomy may be indicated. Again, this would have been determined during examination of the patient well before the scheduled surgery. Assuming a tonsillectomy is needed, the surgeon performs the tonsillectomy and then applies the appropriate hemostatic treatment as noted in the eighth step 24 of the flowchart. The surgeon may elect to apply a hemostatic gel, e.g., Surgiflo® using the conventional applicator, or perhaps a hemostatic sheet material, as deemed appropriate by the surgeon.

In the example of the present disclosure, each of the possible operations of the method is generally described, but it should be understood that it would be extremely rare for any given patient to require all of the operations. In any event, should the pre-surgical examination of the patient indicate that the adenoids should be removed, an adenoidectomy would next be performed, generally as indicated by the ninth step 26 of the flowchart, and the incisions or curetted areas treated, generally as indicated by the tenth step 28 of the flowchart. While conventional surgical techniques are used for the adenoidectomy (and other surgical procedures described herein), the method of closing the incision or curetted area relics upon the application of a hemostatic treatment in the form of a hemostatic gel or hemostatic sheet material rather than the use of sutures, as conventionally performed. This results in significant reduction of trauma for the patient, allowing all of the indicated operations to be performed in a single operating session.

When each of the selected oral operations has been performed, the oral cavity is packed conventionally with hemostatic packs of cotton, gauze, and/or other suitable materials as required, generally as indicated by the eleventh step 30 of the flowchart. These oral packs remain in place only until all of the operational procedures planned for the operating session have been performed. They are removed immediately following all of the surgeries.

The above-described steps comprise all of the oral operations that may be performed in surgical treatment for obstructive sleep apnea. At this point, the surgeon shifts his or her attention to the nose of the patient and proceeds with a first nasal operation, generally as indicated by the twelfth step 32 of the flowchart. There are only two nasal procedures that might be performed, i.e., septoplasty (to correct a deviated septum) and turbinoplasty (to open the inferior turbinate passages), as indicated in the list of the third step 14 of the flowchart. Assuming that both are to be performed, the surgeon would likely elect to proceed initially with the septoplastic surgery to correct the deviated septum of the patient. This surgery is performed conventionally, with the surgeon applying a post-operative hemostatic treatment as described further above in lieu of conventional sutures or cauterizing of the surgical wound(s), generally as indicated by the thirteenth step 34 of the flowchart.

Assuming that the patient also required turbinoplastic surgery as well, the surgeon would then operate to correct this problem, generally as shown by the fourteenth step 36 of the flowchart. The surgical incision(s) would again be treated with an appropriate hemostatic treatment, e.g., a hemostatic gel applied by means of an appropriate conventional applicator, generally as indicated by the fifteenth step 38 of the flowchart. The nasal cavities may then be packed using conventional hemostatic cotton and/or gauze packing. The packing remains in place for the remainder of the operating session.

At this point only a single possible additional surgical procedure remains of the multiple procedures for correcting a patient's obstructive sleep apnea (OSA) condition. That single additional procedure might be grouped with the oral procedures, but is perhaps more accurately considered as a tracheal procedure. This is the relocation or anterior advancement of the hypoid bone, generally as indicated by the sixteenth step 40 of the flowchart. This bone is located in the upper neck, and is not attached directly to any of the other skeletal structure. As it is somewhat free to move, it may cause some narrowing of the throat and subsequent restriction of airflow during sleep, i.e., sleep apnea. The hypoid bone suspension procedure involves the detachment of the upper tendons from the hypoid bone, allowing the bone to be relocated slightly forwardly of its original location. The bone is then permanently secured to the thyroid cartilage, and hemostatic treatment is applied in the form of hemostatic sheet material (e.g., Surgicel®, or other suitable hemostatic treatment) to complete the surgery, generally as indicated by the seventeenth step 42 of the flowchart.

At this point, the previously placed oral and nasal packs remain in place, with the patient breathing by means of the tracheal tube placed before the operations began. Accordingly, the surgeon removes the oral packing and nasal packing, checking to confirm that bleeding is stopped or at least that no more than some light bleeding may occur, treatable by means of further hemostatic gel or sheet material as appropriate. With all packing removed, the nasal and oral breathing passages of the patient are open and the previously installed tracheal tube may be removed, generally as indicated by the eighteenth step 44 of the flowchart. The patient may then be revived to consciousness and monitored during patient recovery, generally as indicated by the final or nineteenth step 46 of the flowchart. Thus, all of the required medical procedures indicated by preoperative examination of the patient for the treatment of his or her sleep apnea have been accomplished in a single operating session as enabled by the use of hemostatic surgical gel and sheet material in lieu of conventional suturing and/or cauterizing of the surgical incisions and wounds.

It is to be understood that the present invention is not limited to the embodiments described above, but encompasses any and all embodiments within the scope of the following claims.

I claim:

1. A method of performing multiple oral and nasal surgical procedures in a single operating session, comprising the steps of:
(a) evaluating the patient to determine the specific operating procedures needed;
(b) selecting the specific operating procedures, based upon the patient evaluation;
(c) preparing the patient for the selected operating procedures, including placing a tracheal breathing tube in the patient;
(d) performing at least one oral operation;
(e) applying a hemostatic treatment to the incision of the oral operation;
(f) performing at least one nasal operation;
(g) applying a hemostatic treatment to the incision of the nasal operation;
(h) removing any oral and nasal hemostatic packs previously applied during the operations;
(i) removing the previously placed tracheal breathing tube; and
(j) monitoring the recovery of the patient.

2. The method of performing multiple oral and nasal surgical procedures according to claim 1, further comprising the steps of:
(a) providing a hemostatic surgical gel;
(b) preloading an applicator with the hemostatic surgical gel; and
(c) applying the hemostatic surgical gel to the incision of the operation using the applicator.

3. The method of performing multiple oral and nasal surgical procedures according to claim 2, further comprising the step of providing the hemostatic surgical gel with analgesic and antibiotic properties.

4. The method of performing multiple oral and nasal surgical procedures according to claim 1, further comprising the steps of:
(a) providing a hemostatic sheet material; and
(b) applying the hemostatic sheet material to the incision of the operation.

5. The method of performing multiple oral and nasal surgical procedures according to claim 4, further comprising the step of providing the hemostatic sheet material with analgesic and antibiotic properties.

6. The method of performing multiple oral and nasal surgical procedures according to claim 1, further comprising the step of selecting a plurality of surgical procedures from the group of surgical procedures consisting of septoplasty, tonsillectomy, adenoidectomy, nasal turbinate surgery, hypoid suspension surgery, and palatal surgery.

7. A method of performing multiple oral and nasal surgical procedures in a single operating session, comprising the steps of:
(a) evaluating the patient to determine the specific operating procedures needed;
(b) selecting the specific operating procedures, based upon the patient evaluation;
(c) preparing the patient for the selected operating procedures, including placing a tracheal breathing tube in the patient;
(d) providing a hemostatic surgical gel;
(e) providing an applicator and preloading the applicator with the hemostatic surgical gel;
(f) performing at least one oral operation;
(g) applying a hemostatic treatment to the incision of the oral operation;
(h) removing any nasal hemostatic packs previously applied;
(i) performing at least one nasal operation;
(j) using the applicator to apply the hemostatic surgical gel to the incision of the nasal operation;
(k) removing any oral hemostatic packs previously applied during the operations;
(l) removing the previously placed tracheal breathing tube; and
(m) monitoring the recovery of the patient.

8. The method of performing multiple oral and nasal surgical procedures according to claim 7, further comprising the step of providing the hemostatic surgical gel with analgesic and antibiotic properties.

9. The method of performing multiple oral and nasal surgical procedures according to claim 7, further comprising the steps of:
(a) providing a hemostatic sheet material; and
(b) applying the hemostatic sheet material to the incision of the operation.

10. The method of performing multiple oral and nasal surgical procedures according to claim 9, further comprising the step of providing the hemostatic sheet material with analgesic and antibiotic properties.

11. The method of performing multiple oral and nasal surgical procedures according to claim 7, further comprising the step of selecting a plurality of surgical procedures from the group of surgical procedures consisting of septoplasty, tonsillectomy, adenoidectomy, nasal turbinate surgery, hypoid suspension surgery, and palatal surgery.

12. A method of performing multiple oral and nasal surgical procedures in a single operating session, comprising the steps of:
(a) evaluating the patient to determine the specific operating procedures needed;
(b) selecting the specific operating procedures, based upon the patient evaluation;
(c) preparing the patient for the selected operating procedures, including placing a tracheal breathing tube in the patient;
(d) performing at least one oral operation;
(e) providing a hemostatic sheet material;
(f) applying the hemostatic sheet material to the incision of the oral operation;
(g) performing at least one nasal operation;
(h) applying a hemostatic treatment to the incision of the nasal operation;
(i) removing any oral and nasal hemostatic packs previously applied during the operations;
(j) removing the previously placed tracheal breathing tube; and
(k) monitoring the recovery of the patient.

13. The method of performing multiple oral and nasal surgical procedures according to claim 12, further comprising the step of providing the hemostatic sheet material with analgesic and antibiotic properties.

14. The method of performing multiple oral and nasal surgical procedures according to claim 12, further comprising the steps of:
(a) providing a hemostatic surgical gel;
(b) preloading an applicator with the hemostatic surgical gel; and
(c) applying the hemostatic surgical gel to the incision of the operation using the applicator.

15. The method of performing multiple oral and nasal surgical procedures according to claim 14, further comprising the step of providing the hemostatic surgical gel with analgesic and antibiotic properties.

16. The method of performing multiple oral and nasal surgical procedures according to claim 12, further comprising the step of selecting a plurality of surgical procedures from the group of surgical procedures consisting of septoplasty, tonsillectomy, adenoidectomy, nasal turbinate surgery, hypoid suspension surgery, and palatal surgery.
